(19) United States
(12) Patent Application Publication
(10) Pub. No.: US 2018/0146919 A1
(43) Pub. Date: May 31, 2018

(54) SYSTEM AND METHOD OF MONITORING CONSUMABLE USE BASED ON CORRELATIONS WITH DIAGNOSTIC TESTING

(71) Applicant: QUANTUM DENTAL TECHNOLOGIES INC., Toronto (CA)

(72) Inventors: Stephen ABRAMS, Toronto (CA); Josh SILVERTOWN, Toronto (CA); Michael WAINBERG, Toronto (CA); Koneswaran S. SIVAGURUNATHAN, Scarborough (CA)

(21) Appl. No.: 15/580,187

(22) PCT Filed: Jun. 16, 2016
(86) PCT No.: PCT/CA2016/050700

§ 371 Date: Dec. 6, 2017

Related U.S. Application Data
(60) Provisional application No. 62/180,218, filed on Jun. 16, 2015.

(51) Int. Cl. A61B 5/00; G16H 40/20; G06F 17/18; G16H 40/40

(52) U.S. Cl. CPC A61B 5/4833; A61B 5/7275; G16H 40/40; G16H 40/20; G06F 17/18; A61B 5/4547

(57) ABSTRACT

The present disclosure provided example methods for estimating the rate of use of a consumable that is employed during the treatment of a medical condition, where rate of use is estimated based on a pre-determined correlation between diagnostic measurements and use of the consumable. In one example embodiment, the estimated rate of use, as determined using the correlation and diagnostic data, may be compared with a measured rate of use, such as a rate of supply of the consumable. These rates of use may be compared in order to estimate whether or not the consumable is being used as expected based on the diagnostic data.

### CROSS-REFERENCE TO RELATED APPLICATION

This application claims priority to U.S. Provisional Application No. 62/180,218, titled “SYSTEM AND METHOD OF MONITORING CONSUMABLE USE BASED ON CORRELATIONS WITH DIAGNOSTIC TESTING” and filed on Jun. 16, 2015, the entire contents of which is incorporated herein by reference.

### BACKGROUND

The present disclosure relates to the monitoring of consumables associated with the treatment of a medical condition.

Diagnostic medical devices are employed in a number of different settings for the collection of diagnostic measurements for the diagnosis, or as an aid in diagnosis and/or monitoring of a medical condition or medical conditions. Medical consumables are often employed during the treatment of the medical condition, and use of a given consumable is typically dependent on the presence or absence of the medical condition, and/or the severity of the medical condition.

### SUMMARY

The present disclosure provided example methods for estimating the rate of use of a consumable that is employed during the treatment of a medical condition, where rate of use is estimated based on a pre-determined correlation between diagnostic measurements and use of the consumable. In one example embodiment, the estimated rate of use, as determined using the correlation and diagnostic data, may be compared with a measured rate of use, such as a rate of supply of the consumable. These rates of use may be compared in order to estimate whether or not the consumable is being used as expected based on the diagnostic data.

Accordingly, in one aspect, there is provided a computer-implemented method of estimating a rate of use of a consumable, wherein the consumable is employed for the treatment of a medical condition, and wherein a presence and/or severity of the medical condition may be determined, at least in part, using diagnostic measurements made with a diagnostic medical device, the method comprising:

obtaining a pre-determined correlation establishing a predictive relationship between diagnostic measurements obtained with the diagnostic medical device and use of the consumable;

obtaining, from the diagnostic medical device, diagnostic data pertaining to one or more diagnostic measurements;

employing the pre-determined correlation to calculate, based on the diagnostic data, a first estimated rate of use of the consumable;

obtaining consumption data associated with measured use or measured supply of the consumable during a time interval;

determining, based on the consumption data, a second estimated rate of use of the consumable; and

generating output involving a comparison of the first estimated rate of use of the consumable to the second estimated rate of use of the consumable.

In another aspect, there is provided a computer-implemented method of estimating a rate of use of a consumable, wherein the consumable is employed for treatment of a medical condition, and wherein a presence and/or severity of the medical condition may be determined, at least in part, using diagnostic measurements made with a diagnostic medical device, the method comprising:

obtaining a pre-determined correlation establishing a predictive relationship between diagnostic measurements obtained with the diagnostic medical device and use of the consumable;

obtaining, from the diagnostic medical device, diagnostic data pertaining to one or more diagnostic measurements;

employing the pre-determined correlation to calculate, based on the diagnostic data, an estimated rate of use of the consumable.

A further understanding of the functional and advantageous aspects of the disclosure can be realized by reference to the following detailed description and drawings.

### BRIEF DESCRIPTION OF THE DRAWINGS

Embodiments will now be described, by way of example only, with reference to the drawings, in which:

FIG. 1 shows a flow chart illustrating an example method of monitoring the use of a consumable based on diagnostic data obtained from a diagnostic medical device.

FIG. 2 is a table illustrating various example diagnostic medical devices or tests, where each diagnostic medical device or test may be employed, at least in part, for the diagnosis or monitoring of an associated medical condition, and where treatment of the medical condition involves the use of a consumable.

FIGS. 3A-3C are system diagrams illustrating various example systems for monitoring consumable use based on diagnostic data from a diagnostic medical device.

FIG. 4 is a block diagram illustrating an example implementation of a diagnostic medical device.

FIG. 5A shows an example correlation between measurements of a diagnostic medical device and the use of a consumable, as part of the recommended treatment.

FIG. 5B contains a list a series of conditions that would define the risk for a patient developing caries. In an example implementation, the combination of one or more of these risk factors with the Canary Number reading from a tooth surface can be employed to generate one or more treatment recommendations.

FIG. 6A shows an association between measurements made by an example oral health diagnostic device (The Canary System™), with its measurement scale (The Canary number) and a severity of tooth decay.

FIG. 6B shows a treatment guide demonstrating an example correlation between measurements of the example oral health diagnostic device (The Canary System™) and its treatment scale (The Canary number) and the recommended treatment of the oral health condition.

FIG. 6C shows the distribution of detected Canary Numbers on a particular surface on a particular group of teeth for a group of patients from a dental practice in two age ranges.

FIG. 7 shows photographs of a tooth from a selected patient overlaid with The Canary numbers obtained from diagnostic measurements at two different dates, demonstrating the detection of the therapeutic impact of the treatment which is not seen visually.

FIG. 8 is a graphical representation of the change in maximum Canary Number on a particular tooth surface over time.

FIG. 9 is a graphical representation of the number of times fluoride varnish, a consumable was recommended by a group of dental offices using The Canary System, where the recommendation is made based on a correlation (treatment guide) between diagnostic measurements made with the Canary System and use of the consumable.

FIG. 10 is a graphical representation of the Canary Number measurements by age group and Canary Number across a large number of offices.

### DETAILED DESCRIPTION

Various embodiments and aspects of the disclosure will be described with reference to details discussed below. The following description and drawings are illustrative of the disclosure and are not to be construed as limiting the disclosure. Numerous specific details are described to provide a thorough understanding of various embodiments of the present disclosure. However, in certain instances, well-known or conventional details are not described in order to provide a concise discussion of embodiments of the present disclosure.

As used herein, the terms “comprises” and “comprising” are to be construed as being inclusive and open ended, and not exclusive. Specifically, when used in the specification and claims, the terms “comprises” and “comprising” and variations thereof mean the specified features, steps or components are included. These terms are not to be interpreted to exclude the presence of other features, steps or components.

As used herein, the term “exemplary” means “serving as an example, instance, or illustration,” and should not be construed as preferred or advantageous over other configurations disclosed herein.

As used herein, the term “medical condition” is meant to cover health related conditions, including conditions associated with physical health, mental health, and nutritional health. A non-limiting example of physical health is oral health.

As used herein, the terms “about” and “approximately” are meant to cover variations that may exist in the upper and lower limits of the ranges of values, such as variations in properties, parameters, and dimensions. Unless otherwise specified, the terms “about” and “approximately” mean plus or minus 25 percent or less.

It is to be understood that unless otherwise specified, any specified range or group is as a shorthand way of referring to each and every member of a range or group individually, as well as each and every possible sub-range or sub-group encompassed therein and similarly with respect to any sub-ranges or sub-groups therein. Unless otherwise specified, the present disclosure relates to and explicitly incorporates each and every specific member and combination of sub-ranges or sub-groups.

As used herein, the term “on the order of”, when used in conjunction with a quantity or parameter, refers to a range spanning approximately one tenth to ten times the stated quantity or parameter.

Unless defined otherwise, all technical and scientific terms used herein are intended to have the same meaning as commonly understood to one of ordinary skill in the art. Unless otherwise indicated, such as through context, as used herein, the following terms are intended to have the following meanings:

As used herein, the phrase “database” is used in its broadest sense to refer to a digital collection of data or information stored on a data store such as a computers main memory, on one or more a hard disks, on one or more solid-state or flash disks, or on other relevant computer-based storage systems. In some embodiments, a database may be stored on, or interfaced with, a server, such that the database can be accessed by a remote computing device (for example, a mobile computing device) through a data network.

Diagnostic medical devices are employed in a number of different settings for the collection of diagnostic measurements for the diagnosis, or as an aid in diagnosis and/or monitoring of a medical condition or medical conditions. Medical consumables are employed during the treatment of the medical condition, and use of a given consumable is typically dependent on the presence or absence of the medical condition, and/or the severity of the medical condition. The use of a consumable and the measurements obtained from a diagnostic medical device are typically thought of as independent and lacking association.

The use of a consumable and the measurements obtained from a diagnostic medical device are typically thought of as independent and lacking association. However, in some clinical contexts, the diagnostic measurements (made by a diagnostic medical device) and the use of a consumable may both dependent on the presence and/or severity of the medical condition when both the diagnostic medical device and the consumable are associated with a medical condition. In such cases, the rate of use of a consumable may be estimated based on diagnostic data obtained from a diagnostic medical device. This may be achieved, for example, when a correlation or relationship is known between the diagnostic measurements and use of the consumable is known or measureable. This correlation may relate the use of a consumable to a diagnostic data, and/or the rate of use of the consumable to the diagnostic data. As described below, the correlation may additionally or alternatively involve a risk factor associated with a patient.

Accordingly, various aspects of the present disclosure employ a relationship or correlation between diagnostic measurements of a diagnostic medical device and the use of a consumable, where the diagnostic medical device and the consumable are associated with the diagnosis (e.g. as an aid in the diagnosis) (and/or monitoring) of, and treatment of, a medical condition, respectively. As described below, this correlation may take various forms, such as, for example, a look-up table relating possible values of diagnostic measurements to use, or rate of use, of a consumable or the probability of use, or rate of use, of a consumable, or for example, a mathematical relationship (e.g. an equation of a set of coefficients describing an equation) relating diagnostic measurements to use, or rate of use, of a consumable or the probability of use, or rate of use, of a consumable. In some example implementations, a diagnostic measurement having a value exceeding a pre-selected value may trigger the commencement of a treatment plan involving the repeated use of a consumable (e.g. the use of the consumable at a fixed interval, such as twice a day). In embodiments in which diagnostic data is correlated with the use of a consumable, diagnostic data pertaining to diagnostic measurements made over a time interval may be employed to determine, via the correlation, an estimated rate of use of the consumable.

It will be understood that a rate of use may be expressed in several different forms. For example, a rate of use may be expressed as a time-dependent value, for example, a quantity divided by time (e.g. a consumption rate of 50 units/week, or expressed as a quantity within a given time frame (e.g. 100 units were used within a two-week time frame).

The ability to estimate or predict the rate of use of a consumable may be beneficial in determining whether or not a consumable is being used as expected and/or directed by a given patient or within a clinical setting. For example, a consumable may be employed in a medical clinic, applied or placed in the area of concern (or sold by the medical clinic or another merchant such as a pharmacy) for the treatment of the medical condition, and a vendor or supplier of the consumable may wish to determine whether or not the consumable is being used as expected for the treatment of the medical condition. For example, the vendor may wish to know if a clinician within the medical clinic is using the consumable at a rate that would be expected based on the number of clinical cases that are presented to the clinic with the medical condition diagnosed by the medical device. The vendor may be interested in understanding, for example, if the consumable is being underused or overused based on the clinical caseload at the medical clinic with the medical condition diagnosed by the medical device. In the past, the vendor would likely need to rely on verbal interactions with the clinicians at the medical clinic in order to obtain determine whether or not a consumable is being used effectively within a clinical setting. At times, the clinician may not recall or have recorded the instructions or recall how to assess if the consumable is being used effectively and in the proper manner to treat a medical condition.

Accordingly, in some example embodiments of the present disclosure, an estimate of the rate of use of a consumable may be determined by obtaining diagnostic data from a diagnostic medical device, and using the aforementioned relationship or correlation between the diagnostic device measurements and the use, or rate of use, of the consumable in order to predict or estimate a rate of use of the consumable. This estimated rate of use may then be compared with a second estimate of the rate of use of the consumable, where the second estimate is based on known or estimated supply or inventory levels of the consumable. The results of this comparison may then be presented in order to allow for the determination of whether or not the consumable is being used at a rate that is consistent with the rate estimated based on the correlation with the diagnostic data and/or optimal or appropriate treatment of the medical condition.

The diagnostic medical device may be any device that collects or measures diagnostic measurements that may be employed for the diagnosis and/or monitoring of one or more medical conditions. In various non-limiting examples, the diagnostic data may be processed to perform a diagnosis of a medical condition, to assist in the diagnosis of a medical condition, or for the monitoring of a medical condition. A diagnostic medical device need not provide a diagnosis of a medical condition, but may instead measure or generate diagnostic data that can be employed for the determination or assessment of a medical condition. For example, a diagnosis of a medical condition may be made by a medical professional that analyzes or interprets the diagnostic data obtained from the diagnostic medical device.

The diagnostic medical device data may be combined with various risk factors to determine the severity of the disease and or recommended treatment and or the type of consumable to use and how often it is to be used. The diagnostic medical device may gather data at one or more time frames and combine this data with various risk factors for the disease, to develop a diagnosis and frequency for use of the medical consumable. The diagnostic data may also gather data on not only the diagnosis but the recommended treatment for the particular medical condition.

In one non-limiting example, a diagnostic medical device may be a complex device residing within a hospital, such as a magnetic resonance scanner, or in another example, a diagnostic medical device may be a device employed within a medical clinic, such as an emergency ward, a physician's office, a walk-in clinic, a dental clinic, a pharmacy, a physiotherapy clinic, a chiropractor clinic, a sports medicine clinic, a workplace medical facility, or other ambulatory clinics. In another example, a diagnostic medical device may be a home-use device, such as a blood glucose monitor or a blood pressure monitor. A diagnostic device may be an in-vitro device, or, for example, an in-vivo device that is implanted, or at least partially implanted, within a patient.

A consumable may be used by a medical practitioner for the treatment of a medical condition of a patient, or may be employed directly by the patient, or may be employed by an untrained person for the treatment of a medical condition experienced by another person. For example, the consumable may be a pharmaceutical that is provided to a patient for the treatment of a medical condition. Another example of a consumable is therapeutic transdermal patch that may be provided to a patient for the treatment of a medical condition, where the presence or severity of the medical condition can be determined based on measurements from a diagnostic medical device. Another example of a consumable is a compound that the patient may apply to a tooth or other oral tissues to treat an oral health condition such as periodontal disease, erosion, parafunction or caries. Upon detection of a medical condition, a consumable may be used a single time, or may be used in a repeated manner, for example, at a fixed frequency of use, optionally for a prescribed time interval.

A consumable, as described herein, may be contrasted with a “diagnostic consumable” such as a diagnostic reagent, cartridge, or sheath that is employed by the diagnostic device during a diagnostic measurement. Such “diagnostic consumables” are employed by the diagnostic medical device regardless of the outcome of the diagnostic measurement, in contrast with the consumables described herein, for which their use is associated with the outcome of a diagnostic measurement. For example, and oral health consumable may be employed for treatment including remineralization of dental tissue and/or reduction in oral bacteria and or stabilization of oral tissues after exposure to a noxious substance.

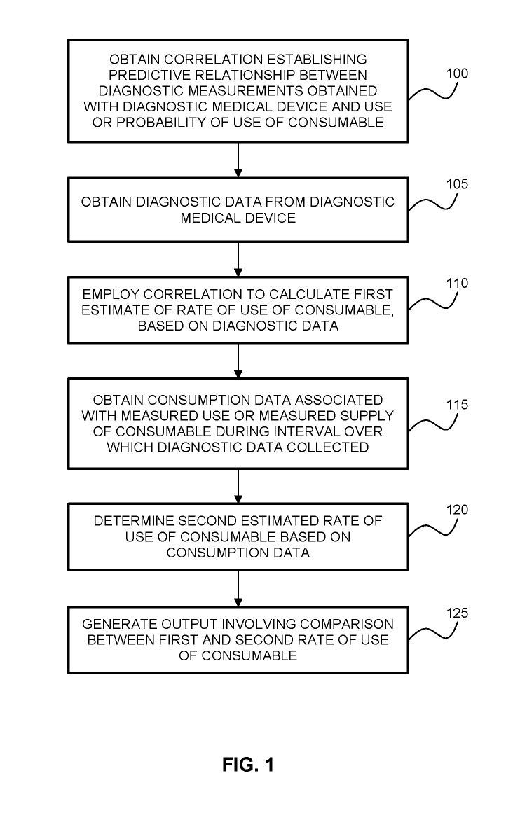

Referring now to FIG. 1, a flow chart is provided illustrating an example method of estimating a rate of use of a consumable, wherein the consumable is employed for the treatment of a medical condition, and wherein a presence and/or severity of the medical condition may be determined, at least in part, using diagnostic measurements made with a diagnostic medical device. In some non-limiting embodiments, at least a portion of the severity and/or presence of the medical condition may be determined by assessing known risk factors and or combining risk factor information with the information from the diagnostic medical device or diagnostic medical devices. In some example embodiments, the diagnostic data may also include risk factor data, such that the diagnostic data combines data obtained based on one or more measurements made with the diagnostic medical device with risk factor data.

At step 100, a correlation is obtained establishing a predictive relationship between diagnostic measurements of a diagnostic medical device and the use, or rate of use, or probability of use, or rate of use, of a consumable, where the diagnostic medical device may be employed, at least in part, for the diagnosis or monitoring of a medical condition, and where the consumable is employed for or during the treatment of the medical condition. Example forms of the correlation, and methods of generating or establishing the correlation, are provided within this disclosure.

In step 105, diagnostic data is obtained from the diagnostic medical device. The diagnostic data may be obtained for one or more patients. For any given patient, the diagnostic data may be associated with a single measurement (e.g. if the correlation establishes a rate of use of the consumable based on a diagnostic measurement) or a plurality of diagnostic measurements (e.g. measured over a time interval, if the correlation relates use of the consumable to a diagnostic measurement). The diagnostic data may be collected from multiple patients over a time interval.

For example, if the diagnostic medical device obtains a measurement for in step 105 a given patient which indicates that a consumable needs to be employed (e.g. placed, used or consumed) to restore function or to remove infected tissue, then the rate of use of the consumable may be estimated based on diagnostic data from the measurement (using the correlation). The diagnostic medical device may then be used to examine, over time, the integrity of the therapy.

As described below, the diagnostic measurements may be made using one or more diagnostic devices, at one or more locations or clinical settings, for one or more patients.

The correlation is then employed, in step 110, to calculate a first estimate of the rate of use of the consumable during a time interval, based on the diagnostic data. For example, the correlation may be employed to provide an estimate, based on the diagnostic data, of how many times the consumable would likely be used during the time interval. This result may then be expressed as a first estimated rate of use of the consumable. The first estimated rate of use of the consumable may be expressed as a single rate associated with the time interval, or may be expressed as a plurality of rates at different times during the interval, such that a time-dependent rate can be established.

Consumption data pertaining to the time interval is then obtained at step 115, the consumption data relating to the measured use or measured supply of the consumable. As described below, this data may be obtained, for example, from a vendor database having data pertaining to the time-dependent supply of the consumable. In another example embodiment, the diagnostic medical device may be located in a medical clinic, and if access is available to a practice management system associated with the clinic, and if the practice management system includes inventory data pertaining to the consumable, the consumption data may be obtained based on the inventory data. In another example, the consumption data may be obtained from a review of electronic invoices from one vendor or online review of invoices obtained by using electronic methods for payment to single or multiple vendors. As the payments are made with an electronic device, the data from this electronic payment system is provided to the vendor to show the purchase of the consumable by the medical clinic, medical provider or patient.

In another example embodiment, an electronic device associated with the use of the consumable may be employed to track and record use, and thereby generate consumption data. For example, an oral health diagnostic device may detect one or more areas of tooth decay or caries in one individual and the medical provider may provide a treatment plan that involves the use of a toothpaste and/or other medical consumable applied on a regular basis. In this example, the patient may be given an electric toothpaste applicator with Wifi communication capabilities that records the use of the toothpaste (the consumable). Alternatively, the patient may be provided with an electric toothbrush with Wi-Fi capabilities that registers the time and frequency of brushing, which can be associated with a rate of use of the toothpaste. This data therefore provides consumption/use data associated with the use of the consumable. The consumption/use data may be stored on a remote server (e.g. in the cloud), where the medical provider, and/or the consumable vendor (and/or vendor of the consumable application device—i.e. the toothbrush in the present example) can review and analyze the data. Ongoing periodic measurements using the oral health diagnostic device, combined with the consumption data of the toothpaste or another medical consumable, may then be employed according to the present example embodiments in order to correlate actual or measured use with use estimated via the diagnostic data.

This information may be employed, for example, by the medical provider to determine compliance of the patient with the treatment plan. If the diagnostic device does not detect a decrease in the size of the decay or the lesion or lesions increase in size or more lesions are detected on other teeth then this is an indication that the consumable was not able to treat the condition or the patient was not compliant or using the consumable properly or the tooth paste application properly.

In embodiments in which the consumable is applied at the same location or setting as the diagnostic medical device, consumption data may be collected by the diagnostic medical device and the consumption data may thus be obtained by communicating with the diagnostic medical (or with a computing device connected to the diagnostic medical device). For example, the diagnostic medical device may be equipped with a reading and/or scanning device that detects and records the presence of a consumable, such that a patient or medical provided can scan the consumable with the diagnostic medical device prior to, during, or after use of the consumable. Example passive modalities for consumable use tracking include optical codes (e.g. 1D or 2D barcodes), optical image or text recognition, and radio-frequency identification tags. Alternatively, if the consumable is used by a powered device (e.g. a powered injection device, or smart medication dispensing device, or an electronic applicator of a consumable), then wireless communication (e.g. near-field communication, Bluetooth or Wi-Fi) may be employed to communicate consumable use information to the diagnostic medical device.

The consumption data may then be processed to determine a second estimated rate of use of the consumable, as shown at step 120. For example, if the consumption data is based on the supply of the consumable to a medical clinic, the second estimated rate of use of the consumable may be calculated based on the quantity of the consumable (optionally the time-dependent quantity) supplied to the medical clinic during the time interval (or over a suitable time duration before the time interval that would be associated with consumption of the consumable during the time interval).

Having determined the first and second estimated rate of consumption of the consumable, the output may be generated that involves a comparison of the two rates, as shown at step 125. For example, in one implementation, the output may be a report describing the first and second estimated rates of use of the consumable. In implementations in which the first and second rates are generated as time-dependent rates (as opposed to single, discrete values), the first and second rates may be shown in a graph or chart, so that the similarities or differences between the rates may be visually examined.

In some example embodiments, output such as an alert or warning may be generated when the second rate differs from the first rate by a pre-selected threshold amount. This alert may be generated and provided to any one or more of the diagnostic vendor/supplier, the consumable vendor/supplier, regulatory body that ensures proper use of the consumable and the clinical setting. In one example embodiment, the threshold may be based on a clinical guideline associated with the use of the consumable in relation to value of the diagnostic measurement. In another example embodiment, the threshold may be based on a contractual obligation involving the use of a companion diagnostic and a consumable.

In one example, an alert or warning is given the staff at the medical clinic if the device detects that the medical condition has worsened and there is need to increase the frequency or amount of consumable used.

In one example application of the aforementioned method, a consumable vendor may partner with a diagnostics vendor, enabling the determination a first estimated rate of use of the consumable based on the diagnostic data collected by the diagnostic vendor (using the correlation described above) and the determination of a second estimated rate of use of the consumable based on the supply of the consumable, as monitored by the consumables vendor. These estimated rates may then be compared in order to estimate whether or not the consumable is being used at a rate that is consistent with the diagnostic data, and/or achieving an appropriate, expected, or optimal medical outcome. For example, the rates may be compared in order to infer an underutilization or an overutilization of the consumable.

This example method may be employed, for example, to detect when a medical clinic reduces its use of the consumable relative to an expected rate of use. For example, if the second estimated rate of use of the consumable (determined, for example, using supply or inventory data) differs from the first estimated rate of use of the consumable by a pre-selected threshold, an inference may be made that the medical clinic has is reducing, on a per-patient level, its rate of use of the consumable. The vendor may suspect, in such a case that the medical clinic may have switched to an alternative consumable, or that the medical clinic is reducing or changing its treatment practices. In either case, the vendor may make inquiries to the medical clinic in order to try to determine the cause of the reduction and/or to attempt to restore use and sales of the consumable.

For example, an alert or warning is given to a regulatory body if the device detects that the medical condition has worsened in one patient or a group of patients using the consumable at the same frequency. In another variation, the regulatory body would receive a warning if the device detects that a medical condition has worsened in one or more patients using the consumable at a lower than recommended frequency or higher than recommended frequency. The regulatory body could then contact the medical clinic to ensure that the consumable is used properly to treat the appropriate condition.

In one example embodiment, when the second estimated rate differs from the first estimated rate by an amount that exceeds a pre-selected threshold, further processing may be performed in order to provide a determination or estimate as to whether or not a substitute consumable is being used in place of the consumable. The diagnostic data may be processed on a per-patient basis for one or more patients, in order to determine a trend in the time-dependence of the diagnostic measurements for the one or more patients during the time interval. If the trend (e.g. a trend associated with a single patient, or an average trend associated with a plurality of patients) is indicative of an improvement in oral health or overall health, despite the inferred underutilization of the consumable, a determination may be made that a substitute consumable may be displacing the use of the consumable. It is noted that this example embodiment may be implemented in a clinical setting (one or more medical clinics or hospitals) or in a non-clinical setting (e.g. home use or portable use involving patient testing and treatment by non-medically trained individuals such as the patient).

It is noted that the example methods disclosed above pertain to comparative monitoring of the rate of use of a consumable, in contrast to merely monitoring the actual rate of use of a consumable. For example, the rate of use of a consumable by a medical clinic, as determined based on supply or inventory levels, may be decreasing with time. One may naively conclude that the clinic is reducing its use of the consumable. However, it may merely be the case that the patient load of the clinic is decreasing or changing with time, and the medical clinic may be maintaining a constant use of the consumable of a per-patient basis. In this case, the aforementioned example comparative monitoring methods, employing the collection of diagnostic data and the utilizing a correlation between the diagnostic measurements and the use of the consumable, would show the first and second rates of use of the consumable as both decreasing, allowing the vendor to conclude that the actual use patterns of the consumable by the medical clinic has not changed. In this situation, the vendor could inform the clinic of the change in medical conditions within their respective patient population.

In another example, the comparative monitoring of the rate of consumable consumption may be used to identify patients or groups of patients that are not compliant with the instructions for the use of the consumable. This identification of a patient or group of patients can help in providing additional instruction on the use of the consumable or identify issues and challenges with the consumable that would make it difficult and or challenging for a patient or group of patients to use the consumable for treatment.

The preceding example embodiment involved a partnership between a diagnostics vendor and a consumables vendor. However, in another example embodiment, the consumable vendor and the diagnostics vendor may be one and the same, or may be related thought a relationship such as a subsidiary or joint venture.

As outlined above, in various example embodiments of the present disclosure, a diagnostic medical device is employed to obtain diagnostic measurements that can be employed for the diagnosis of a medical condition, where the use of a consumable for treating the medical condition or during the treatment of the medical condition can be correlated with the diagnostic measurements.

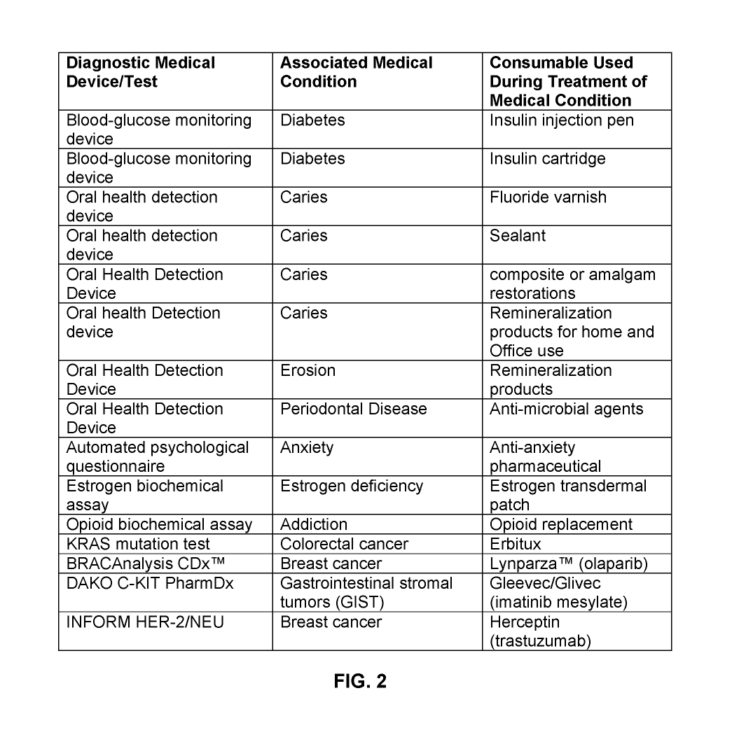

FIG. 2 provides a non-limiting set of examples in which a diagnostic medical device is employed to obtain diagnostic measurements that can be employed for the diagnosis of a medical condition, and where an associated consumable is provided for use during the treatment of the medical condition, and where the use of the consumable depends on the presence or severity of the medical condition. Examples of medical condition include diabetes, cardiovascular disease, periodontal disease, erosion, tempromandibular joint dysfunction, and oral cancer.

A diagnostic medical device may be a “companion” medical diagnostic device, or a “companion diagnostic” that performs, for example, a diagnostic measurement, a test, or an assay that produces a results dependent on the amount of an analyte present in a sample, where the analyte is associated with a medical condition, and where the result is employed to determine a suitable amount of a therapeutic consumable to provide to the patient. FIG. 2 includes some non-limiting examples of companion diagnostics and their associated therapeutic consumable.

In some example embodiments, the diagnostic medical device is employed for the detection of diagnostic data associated with oral health. For example, the diagnostic medical device may be employed for the detection of diagnostic data that may be processed or interpreted for the detection or monitoring of oral health medical conditions such as dental caries. The companion diagnostic medical device may detect the severity and/or presence of dental caries on one or more tooth surfaces with lesions at varying depth and size. In one example implementation, the diagnostic medical device can provide a recommendation of the use of one or more consumables to treat a detected medical condition (optionally based on the severity of the medical condition) or the placement of one or more dental restorations depending upon the depth, volume and size of the lesion as detected by the diagnostic medical device.

In another example, the diagnostic device may detect an oral health condition where a medical consumable is applied or taken internally. For example, if the oral health condition is periodontal disease and the diagnostic device detects and/or measures further deterioration in the gum tissue or its attachment to the tooth or attachment to bone (periodontal pocket), then a recommendation can be made to change either the type of consumable or the frequency of application or ingestion.

In another example, a diagnostic medical device can detect and/or measure caries and a consumable may be applied in a medical clinic and/or by the patient at home in order to reduce the size of the lesion, harden the surface of the lesion or prevent the lesion from growing in size. Over time, with repeat measurements, the diagnostic medical device would detect and measure changes in this area of decay and other areas of decay on all the teeth providing information on the outcomes of the use of a particular consumable.

Although many of the example embodiments described herein refer to a single diagnostic device having an associated single medical condition, and an associated single consumable that is employed during the treatment of the medical condition, such a 1:1:1 relationship is merely provided for illustrative purposes. For example, a single diagnostic medical device may have two or more medical conditions associated therewith. Also, a single medical condition may have multiple consumables associated therewith, and the consumables may be applied or taken or used at varying time intervals. In another example, there may be multiple diagnostic devices (or measurements, e.g. multiplexed measurements, tests or assays) for a single medical condition, and their output may be combined to provide a single measure or diagnosis and treatment recommendation.

As described above, many of the example embodiments of the present disclosure employ a correlation between measurements of a diagnostic medical device and the use, or rate of use, of a consumable, where the consumable is employed during the treatment of a medical condition, and where the medical condition may be diagnosed and/or monitored at least in part based on the diagnostic measurements. This correlation may be determined according to a wide range of methods, several examples of which are provided herein and described below.

In one example implementation, the correlation may be established by processing historical diagnostic data and historical consumable use data, in order to correlate the use of the consumable, or a probability of use of the consumable, with values of the diagnostic measurements. For example, the correlation may be determined by processing data obtained during a clinical trial. In another example implementation, the correlation may be established by first determining a first correlation between the diagnostic measurement and the presence and/or severity of the medical condition, and also a second correlation between the presence and/or severity of the medical condition with the use or probability of use of the consumable. The first and second correlations may then be combined into a single correlation between the diagnostic measurements and the use or probability of use of the consumable. It will be understood that these example methods of determining the correlation are intended merely as non-limiting illustrative examples.

The aforementioned embodiments show that a correlation may be employed to determine that a given medical condition is not being treated with the use of a consumable, as expected or estimated, based on diagnostic data associated with a first medical condition. In some cases, there may be a linkage between the first medical condition and a second medical condition, such as a linkage between oral health conditions and general health. Accordingly, in such cases, the departure from expected consumable use may be employed to determine the presence and/or risk of the second medical condition. For example, if a patient has periodontal disease and is not being treated with consumables that reduce the periodontal (gum) disease, then it may be determined that their risk for diabetes or control of their diabetes increases.

Although the correlation is described in the example embodiments provided above as a relationship between a diagnostic measurement and a use, or rate of use, or probability of use, or rate of use, of the consumable, the correlation can be based on various forms of diagnostic data and/or results. For example, in one implementation, the correlation can be based on raw unprocessed data, or partially processed data obtained from the diagnostic medical device. Alternatively, the correlation can be based on a diagnostic result obtained by processing raw data from the diagnostic medical device. The diagnostic result may be an intermediate result, value or parameter, or may be an estimated or inferred diagnosis or severity level of the medical condition. The diagnostic result may alternatively provide a risk of a patient contracting a medical condition, where the use of the consumable is correlated with the risk level.

The correlation need not be established between a single diagnostic result and a use or probability of use of the consumable. For example, in an alternative example implementation, the correlation may relate the frequency of occurrence of a binned range of diagnostic measurements or results with a rate of use or probabilistic rate of use of the consumable.

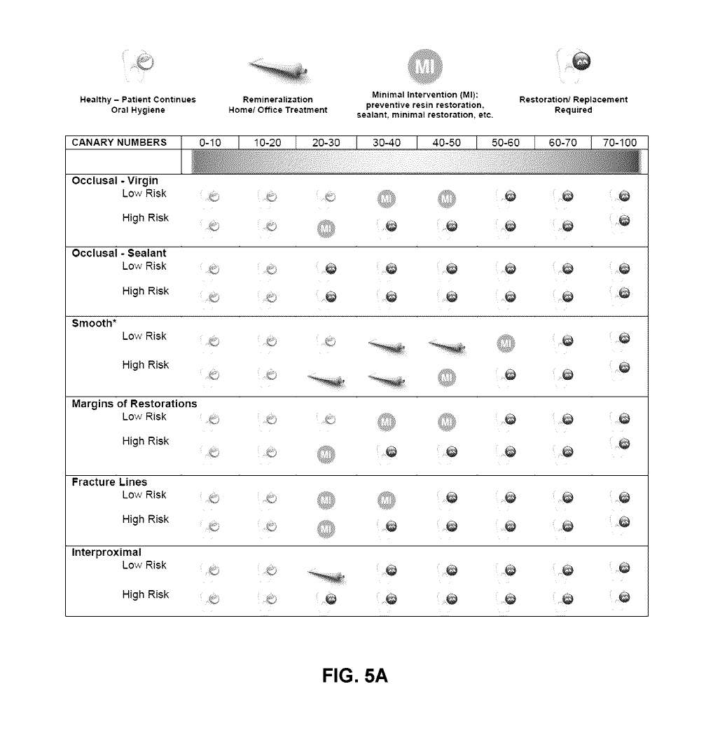

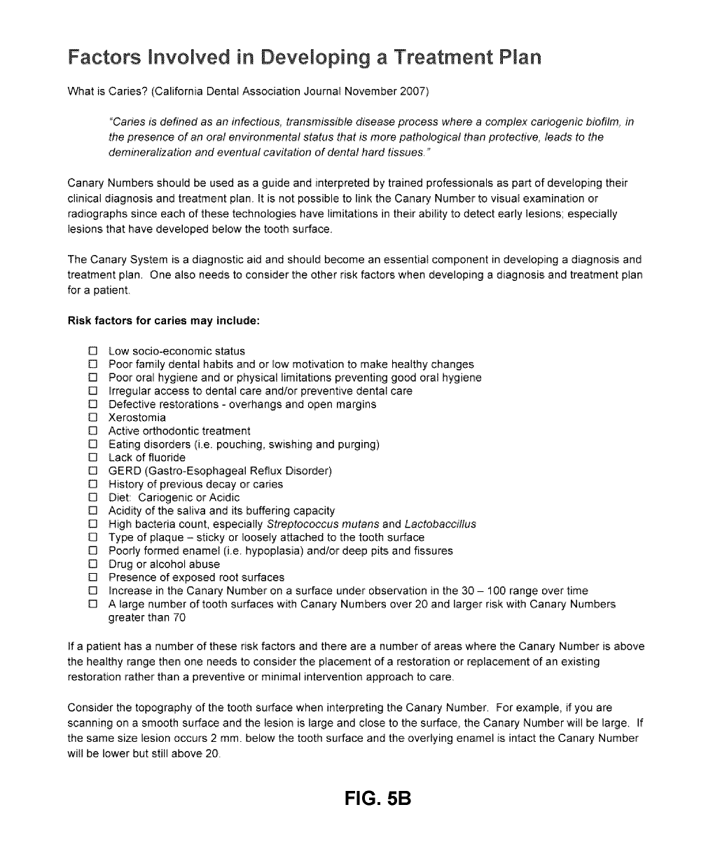

Referring now to FIG. 5, an example chart is provided showing a correlation between measurements of an oral health detection device and the use of a consumable. The oral health detection device employed in this example measures changes in the crystal structure of the tooth using Photothermal Radiometry and ac Luminescence to provide the information on tooth decay or caries. Such devices are described, for example, in U.S. Pat. No. 8,306,608, titled “Method and Apparatus Using Infrared Photothermal Radiometry (PTR) And Modulated Laser Luminescence (LUM) for Diagnostic of Defects in Teeth” and US Patent Application Publication No. 2013/0141558, titled “Handpiece with Integrated Optical System for Photothermal Radiometry and Luminescence Measurements”, both of which are incorporated herein by reference in their entirety.

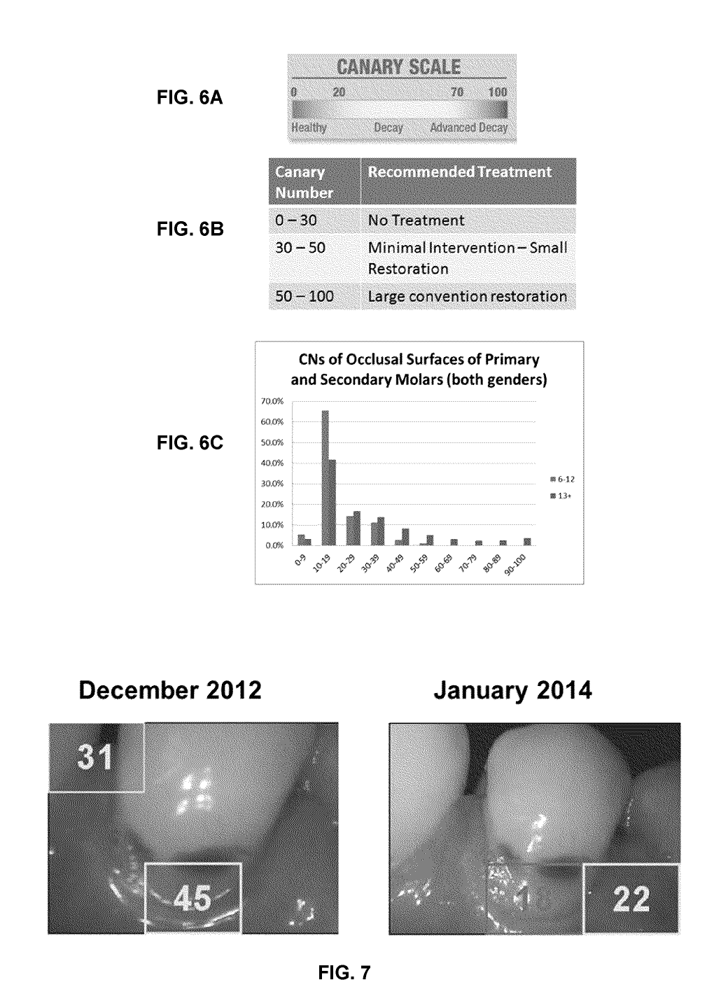

The oral health detection device of the present example is known as the Canary System™, and provides a number referred to as the “Canary Number”, which is related to the oral health status of the patient. FIG. 5B provides a list of possible risk factors that would be part of developing the correct treatment recommendation. For example, FIG. 6A shows the association between measurements made by the Canary System™ (using the Canary number) and the severity of tooth decay. In FIG. 6C, data from a diagnostic oral health detection device (employing photothermal radiation and luminescence detection modalities) was used to detect changes and defects in the crystal structure of teeth at a dental practice (the data was gathered for patients aged 6-12 years and 13 and over). FIG. 6B shows the treatment guide employed to establish a correlation between measurements of the oral health diagnostic device and the recommended treatment of the oral health condition.

Using the correlation provided by the recommended treatment guide, the dental practice was found to have less than 10% of the 13 and over patients that required large filling on their molar teeth. This then provides the vendor, medical clinic or pharmacy with the information on the consumption of a particular consumable including but not limited to a dental filling or crown.

FIG. 7 shows photographs of a tooth from a selected patient overlaid with the Canary numbers obtained from diagnostic measurements at two different dates, demonstrating the detection of the therapeutic impact of the treatment.

In this example, one can see the limitations of using visual examination to determine the health or integrity of a particular tooth surface. The teeth had two brown spots which did not appear to change from December 2012 until January 2014. A group of consumables were used to try to harden or change the brown spots on the tooth, as per a prescribed correlation between the measured Canary number and consumable use. The tooth structure did not show any visual changes over time, but the diagnostic device detected and measured changes in the sub surface structure in response to the use of therapeutic consumables.

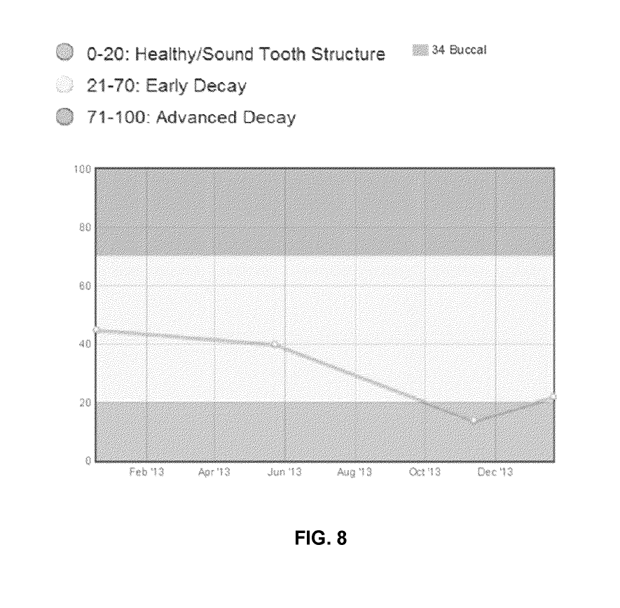

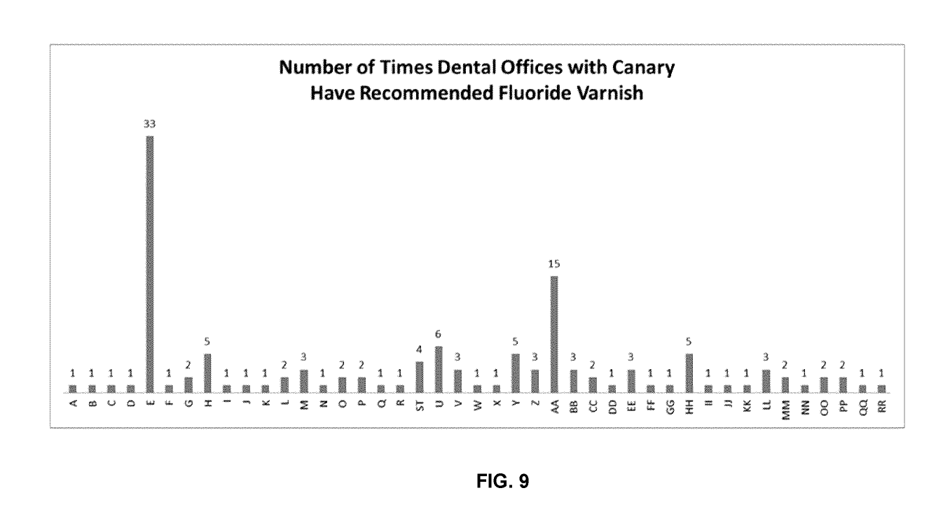

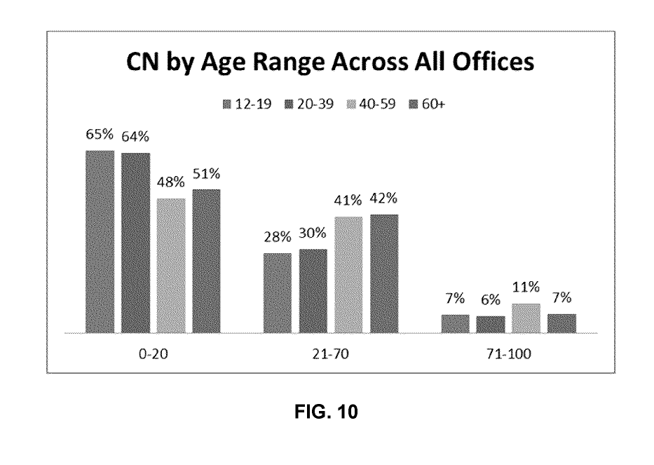

FIG. 8 is a graphical representation of the change in maximum Canary Number on a particular tooth surface over time. FIG. 9 is an example of the number of times a particular therapy is recommended based upon a Canary Number. FIG. 10 is an example of the range of Canary Numbers across a number of medical offices.

Although many of the preceding example embodiments were illustrated in the context of diagnostic measurements being performed at a clinical setting such as one or more hospitals or ambulatory medical clinics, it will be understood that the setting in which the diagnostic measurements are performed may be a near-patient setting, and the diagnostic medical device may be home-use medical device or a portable medical device, such as a glucose monitor. As described in the example systems discussed below, such devices may be interfaced with a remote computing system via a network using wireless technologies such as Wi-Fi and Bluetooth. In such example embodiments involving near-patient testing, the output generated based on the comparison between the first and second rates of use of the consumable (see step 125 in FIG. 1) may be provided to the patient as an alert. For example, the output may be provided as a warning that the patient does not appear to be using the consumable at a clinically relevant rate, as predicted by the measured diagnostic data.

In some example embodiments, the diagnostic data is collected from one or more diagnostic medical devices (e.g. diagnostic analyzers) located at a single medical clinic or hospital (or ward within a hospital). In other example embodiments, the diagnostic data may be collected from a plurality of medical clinics, and/or a plurality of hospitals, and/or a plurality of wards within a hospital. In such cases, the first and second rates (and the output) may be determined on a per-site basis or may be determined on an averaged basis.

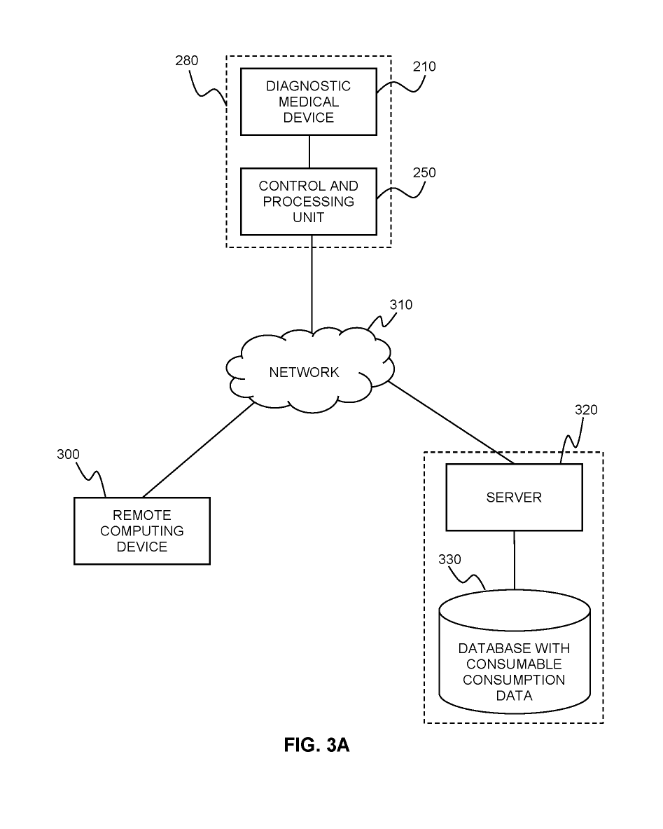

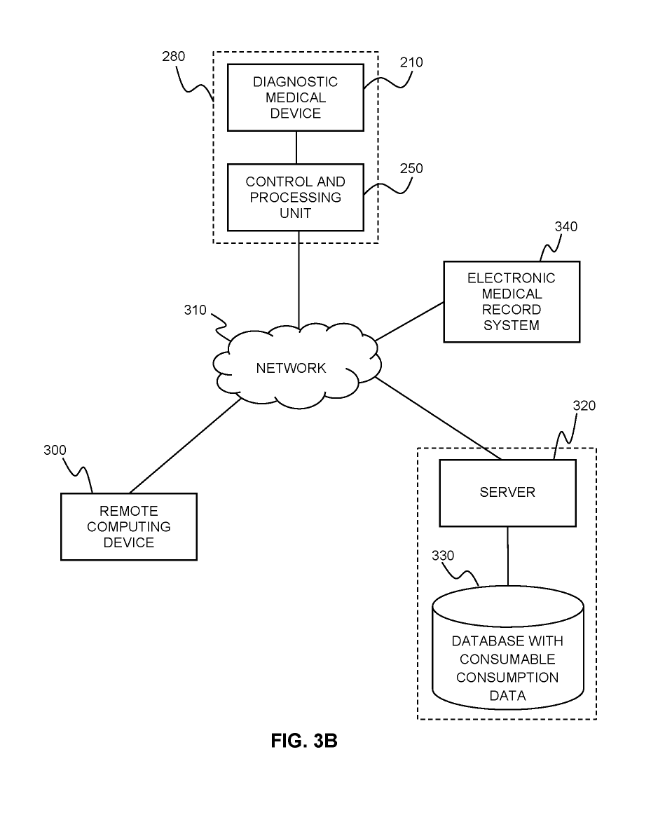

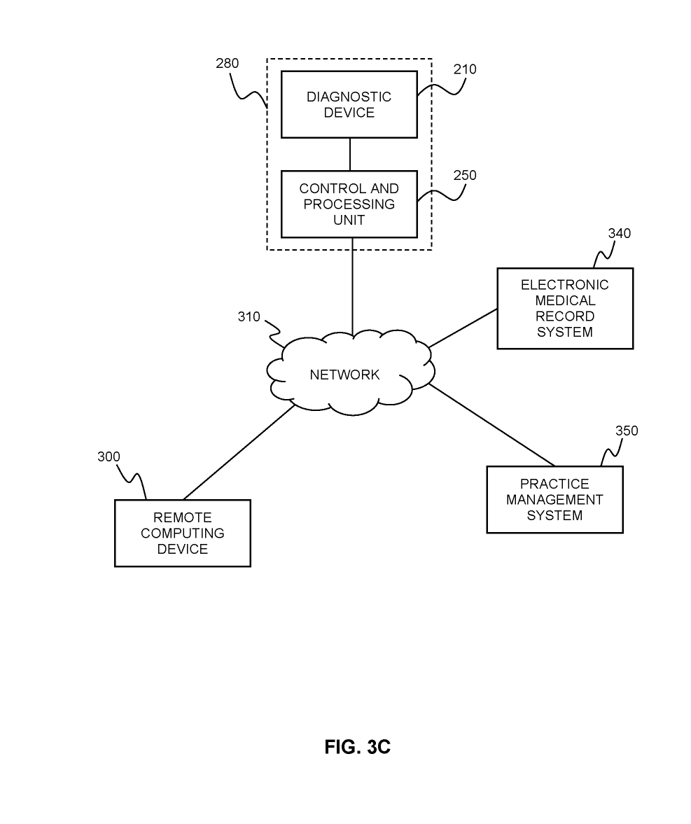

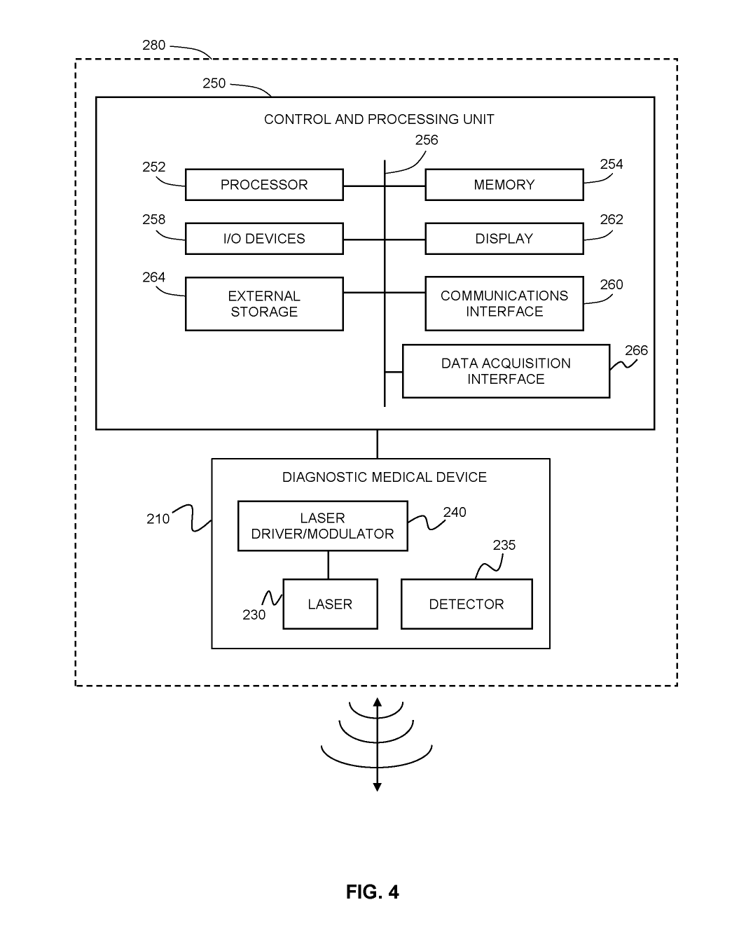

An example system for the monitoring of the use of a consumable based on diagnostic data obtained from a diagnostic medical device is shown in FIG. 3A. The example system includes remote computing device 300 that is interfaced to a diagnostic medical device 210, through network 310, via local control and processing unit 250. As shown in the figure, the diagnostic medical device may be provided as a separate diagnostic device 210 that is interfaced to a local processing unit and control unit 250, or may be provided as an integrated diagnostic medical device that includes embedded processing and communication functionality, as shown at 280. Example implementations of diagnostic medical device 210 and control and processing unit 250 are shown in FIG. 4 and described below.

As shown in FIG. 3A, the remote computing device 300 is connected to the control and processing unit 205 through network, thereby enabling the remote computing device 300 to obtain diagnostic data from diagnostic medical device 210. In one example implementation, the remote computing device 300 may actively poll the control and processing unit 205 associated with the diagnostic medical device 250 in order to obtain the diagnostic data. For example, the remote computing device 300 may transmit a request message to the control and processing unit 250 on a periodic (i.e. intermittent) basis. The control and processing unit 250, upon receipt of a request message, generates a response message containing the diagnostic data, and transmits the response message to the remote computing device 300.

As shown in FIG. 3A, the remote computing device 300 is also connected, through network 310, to a remote server 320, where the remote server 320 includes, or is capable of accessing, the consumption data pertaining to the measured use or supply of the consumable. For example, as shown in the figure, the remote server 320 may be connected to a storage database 330 that includes the consumption data. In one example implementation, the remote server 320 may include a local file storage medium that includes the consumption database. In one example implementation, the remote computing device 300 may actively poll the remote server 320 in order to obtain the consumption data. For example, the remote computing device 300 may transmit a request message to the remote server 320 on a periodic (i.e. intermittent) basis. The remote server 320, upon receipt of a request message, generates a response message containing the consumption data, and transmits the response message to the remote computing device 300.

The remote computing device 300 includes a processor and a memory for executing one or more example methods of the present disclosure. For example, the example methods described above can be partially implemented via hardware logic in a processor and partially using the instructions stored in a memory, where the correlation, and the logic for determining the estimated rate of consumable use based on the diagnostic data, are stored in the memory (or another storage medium) of the remote computing device. Some embodiments are implemented using a processor without additional instructions stored in a memory. Some embodiments are implemented using the instructions stored in a memory for execution by one or more general purpose microprocessors. Thus, the disclosure is not limited to a specific configuration of hardware and/or software.

While some embodiments can be implemented in fully functioning computers and computer systems, various embodiments are capable of being distributed as a computing product in a variety of forms and are capable of being applied regardless of the particular type of machine or computer readable media used to actually effect the distribution.

At least some aspects disclosed can be embodied, at least in part, in software. That is, the techniques may be carried out in a computer system or other data processing system in response to its processor, such as a microprocessor, executing sequences of instructions contained in a memory, such as ROM, volatile RAM, non-volatile memory, cache or a remote storage device.

A computer readable storage medium can be used to store software and data which when executed by a data processing system causes the system to perform various methods. The executable software and data may be stored in various places including for example ROM, volatile RAM, nonvolatile memory and/or cache. Portions of this software and/or data may be stored in any one of these storage devices.

In the example embodiment shown in FIG. 3A, remote computing device 300 may be a remote monitoring computing device that is remotely interfaced with one or more diagnostic medical devices for the remote monitoring thereof. Accordingly, remote computing device 300 may be associated with the vendor or supplier of the diagnostic medical device 210. Server 320 may be associated with the vendor or supplier of the consumable. FIG. 3A may therefore be interpreted to show an example system in which a diagnostic vendor/supplier has partnered with a consumable vendor, such that the data from the diagnostic medical device can be employed to compare the estimated rate of use of the consumable, as determined based on the consumption data collected by the consumable vendor, to the estimated rate of use of the consumable, as predicted based on the correlation between the use of the consumable and the diagnostic data collected from the diagnostic medical device.

In an alternative example embodiment, in which a single vendor provides both the diagnostic medical device and the consumable, the remote computing device 300 and server 320 may be one and the same.

FIG. 3B illustrates an alternative example system embodiment in which the remote computing device 300 is interfaced with an electronic medical records system 340, where the electronic medical records system 340 is interfaced with the control and processing unit 250 via network 310, and where the electronic medical records system 340 collects and stores diagnostic data from the diagnostic medical device 210 (e.g. via active polling, as described above). According to such an example embodiment, remote computing device 300 may communicate with electronic medical records system 340 to obtain the diagnostic data, without needing to communicate with the control and processing unit 250 that is interfaced or embedded within the diagnostic medical device 210. In one example implementation, the remote computing device 300 may actively poll the electronic medical records system 340 in order to obtain the diagnostic data, thereby indirectly obtaining the diagnostic data from the diagnostic medical device. For example, the remote computing device 300 may transmit a request message to the electronic medical records system 340 on a periodic (i.e. intermittent) basis. The electronic medical records system 340, upon receipt of a request message, generates a response message containing the diagnostic data, and transmits the response message to the remote computing device 300. Although the present example embodiment illustrates an example case in which the electronic medical records system 340 is a cloud-based (e.g. software-as-a-service) system, it will be understood that in another example implementation, electronic medical records system 340 may be provided locally on-site with the control and processing unit 250.

FIG. 3C illustrates another non-limiting example system embodiment, in which a practice management system 350 is included, where the practice management system 350 includes inventory data relating to the use of the consumable (for example, by one or more a medical clinics). The remote computing device 300 connects to the practice management system 350 through network 310 and obtains the consumption data by determining the inventory levels over the time interval of interest, without needing to access a remote server associated with the vendor of the consumable. In one example implementation, the remote computing device 300 may actively poll the practice management system 350 in order to obtain the consumption data. For example, the remote computing device 300 may transmit a request message to the practice management system 350 on a periodic (i.e. intermittent) basis. The practice management system 350, upon receipt of a request message, generates a response message containing the consumption data, and transmits the response message to the remote computing device 300.

Although the present example embodiment illustrates an example case in which the practice management system 350 is a cloud-based (e.g. software-as-a-service) system, it will be understood that in another example implementation, practice management system 350 may be provided locally on-site with the control and processing unit 250.

Although FIGS. 3A-C illustrate example embodiments that include a single diagnostic device, it will be understood that in other example embodiments, a plurality of diagnostic medical devices may be connected to the remote computing device 300 for monitoring and consumable use analysis, as described above.

FIG. 4 shows an example implementation of a diagnostic medical device 210, where the example diagnostic medical device 210 includes a laser 230, an optical detector 235, and a laser driver and/or modulator 240, where diagnostic medical device 210 is interfaced with control and processing unit 250. Other examples of diagnostic medical devices will be known to those skilled in the art, such as diagnostic analyzers that are configured to perform biochemical or biomolecular assays on samples, ultrasound-based systems and magnetic resonance imaging systems.

Another example of a diagnostic medical device captures an image of the tooth surface when exposed to one wavelength or different wavelengths of electromagnetic radiation. These images are then used to map or detect changes in the response of the tooth surface under examination over time and relate this to changes on the surface or beneath the surface of the tooth. This data may be analyzed to detect changes over time, and an associated consumable may then be employed for the treatment of a detected oral health condition. The device may be employed to measure the effects of the consumable on the treatment of the caries.

As shown in the example embodiment of FIG. 4, control and processing unit 250 may include a processor 252, a memory 254, a system bus 256, a data acquisition interface 266, one or more input/output devices 258, and a plurality of optional additional devices such as communications interface 260, display 262, and external storage 264. Communications interface 260 may be a wired or wireless transceiver for establishing a connection to a network, as shown in FIGS. 3A-3C. Processor 252 may be employed for tasks such as controlling the operation of the diagnostic medical device, pre-processing (e.g. digitizing) the diagnostic signals received by the diagnostic medical device 210, and controlling, at least in part, the communication of the control and processing unit 250 with externally networked devices.

It is to be understood that the example system shown in the figure is not intended to be limited to the components that may be employed in a given implementation. For example, the system may include one or more additional processors. Furthermore, although FIG. 4 shows separate components forming the diagnostic medical device 210 and the control and processing unit 250, it will be understood that this is merely an example implementation, and that other configurations may be implemented in the alternative. For example, in one alternative implementation, the diagnostic medical device 200 may be directly integrated with the control and processing unit 250, forming integrated diagnostic medical device 280.

The specific embodiments described above have been shown by way of example, and it should be understood that these embodiments may be susceptible to various modifications and alternative forms. It should be further understood that the claims are not intended to be limited to the particular forms disclosed, but rather to cover all modifications, equivalents, and alternatives falling within the spirit and scope of this disclosure.

1. A computer-implemented method of estimating a rate of use of a consumable, wherein the consumable is employed for treatment of a medical condition, and wherein a presence and/or severity of the medical condition may be determined, at least in part, using diagnostic measurements made with a diagnostic medical device, the method comprising:
obtaining a pre-determined correlation establishing a predictive relationship between diagnostic measurements obtained with the diagnostic medical device and use of the consumable;
obtaining, from the diagnostic medical device, diagnostic data pertaining to one or more diagnostic measurements;
employing the pre-determined correlation to calculate, based on the diagnostic data, a first estimated rate of use of the consumable;
obtaining consumption data associated with measured use or measured supply of the consumable during a time interval;
determining, based on the consumption data, a second estimated rate of use of the consumable; and
generating output involving a comparison of the first estimated rate of use of the consumable to the second estimated rate of use of the consumable.

2. The computer-implemented method according to claim 1 wherein the consumption data is obtained by determining inventory levels of the consumable during the time interval.

3. The computer-implemented method according to claim 1 wherein the consumption data is obtained by obtaining supply data pertaining to a time-dependent supply of the consumable prior to, and/or during, the time interval.

4. The computer-implemented method according to claim 3 wherein the supply data is obtained from an external database associated a vendor of the consumable.

5. The computer-implemented method according to claim 1 wherein the diagnostic medical device is configured to detect and record use of the consumable, and wherein the consumption data is obtained from the diagnostic medical device.

6. The computer-implemented method according to claim 1 wherein an additional medical device is employed during use of the consumable, wherein the additional medical device is configured to detect and record use of the consumable, and wherein the consumption data is obtained from the additional medical device.

7. The computer-implemented method according to claim 1 wherein the diagnostic data is obtained from an external database associated a vendor of the diagnostic medical device.

8. The computer-implemented method according to claim 1 wherein the diagnostic data is obtained by communicating with the diagnostic medical device through an external network.

9. The computer-implemented method according to claim 1 wherein the diagnostic data includes risk factor data associated with a risk of contracting the medical condition.

10. The computer-implemented method according to claim 1 wherein the pre-determined correlation is obtained by comparing prior consumable use to prior diagnostic data.

11. The computer-implemented method according to claim 1 wherein the diagnostic medical device is a first diagnostic device residing at a location, and wherein the diagnostic data is associated with the first diagnostic device and with one or more additional diagnostic devices residing at the location.

12. The computer-implemented method according to claim 11 wherein the location is a medical clinic.

13. The computer-implemented method according to claim 12 the medical clinic is a first medical clinic, the method further comprising generating, and comparing, the first estimated rate of use of the consumable and the second estimated rate of use of the consumable for one or more additional medical clinics, and generating the output on a per-clinic basis.

14. The computer-implemented method according to claim 12 the medical clinic is a first medical clinic, the method further comprising generating, and comparing, the first estimated rate of use of the consumable and the second estimated rate of use of the consumable for one or more additional medical clinics, and generating the output based on averaged values of the first estimated rate of use of the consumable and the second estimated rate of use of the consumable for the medical clinics.

15. The computer-implemented method according to claim 12 wherein the range is based on a clinical guideline relating use of the consumable to the diagnostic data.

16. The computer-implemented method according to claim 1 wherein the medical condition is an oral health condition.

17. The computer-implemented method according to claim 16 wherein the diagnostic medical device is an oral health diagnostic device configured to detect the presence of caries, and wherein the consumable is associated with remineralization of dental tissue and/or reduction in oral bacteria and or stabilization of oral tissues after exposure to a noxious substance.

18. The computer-implemented method according to claim 16 wherein the medical condition is selected from the group consisting of diabetes, cardiovascular disease, caries, periodontal disease, erosion, tempromandibular joint dysfunction, parafunctional habits, and oral cancer.

19. The computer-implemented method according to claim 1 wherein the output is an indication of the difference between the first estimated rate of use of the consumable and the second estimated rate of use of the consumable.

20. The computer-implemented method according to claim 1 wherein the output is generated when the second estimated rate of use of the consumable differs from the first estimated rate of use of the consumable by a pre-selected threshold.

21. The computer-implemented method according to claim 20 wherein the diagnostic data pertains to one or more patients, the method further comprising, in the event that the second estimated rate of use of the consumable differs from the first estimated rate of use of the consumable by the pre-selected threshold:
processing the diagnostic data, on a per-patient basis, for one or more patients, to determine a trend associated with medical condition; and
in the event that trend is indicative of an improvement in the medical condition for one or more of the patients, generating additional output warning of possible use of a substitute consumable.

22. The computer-implemented method according to claim 1 wherein the diagnostic data is raw diagnostic data absent of a result, wherein the method further comprises:
processing the diagnostic data to infer a diagnostic result associated with one or more diagnostic tests; and
wherein the pre-determined correlation is between the diagnostic result and use of the consumable.

23. The computer-implemented method according to claim 1 wherein the diagnostic data is raw diagnostic data, and wherein the pre-determined correlation is between raw diagnostic measurements and use of the consumable.

24. The computer-implemented method according to claim 20 wherein the diagnostic data pertains to one or more patients, the method further comprising, in the event that the second estimated rate of use of the consumable differs from the first estimated rate of use of the consumable by the pre-selected threshold:
processing the diagnostic data, on a per-patient basis, for one or more patients, to determine a trend associated with medical condition; and
in the event that the trend is indicative of a lack of improvement in the medical condition, generating additional output warning of potential inappropriate use of the consumable.

25. The computer-implemented method according to claim 24 wherein the warning is communicated to a regulatory body.

26. The computer-implemented method according to claim 24 wherein the warning is communicated to a medical clinic from where the diagnostic data was obtained.

27. The computer-implemented method according to claim 1 wherein the supply data is obtained from an electronic payment device that is configured to capture funds to pay for the consumable along with an identification and quantity of the consumable.

28. The computer-implemented method according to claim 1 wherein the diagnostic data pertains to a diagnostic measurement associated with a single patient.

29. The computer-implemented method according to claim 1 wherein the diagnostic data pertains to a plurality of diagnostic measurements.

30. The computer-implemented method according to claim 29 wherein the plurality of diagnostic measurements are make during the time interval.

31. A computer-implemented method of estimating a rate of use of a consumable, wherein the consumable is employed for treatment of a medical condition, and wherein a presence and/or severity of the medical condition may be determined, at least in part, using diagnostic measurements made with a diagnostic medical device, the method comprising:
obtaining a pre-determined correlation establishing a predictive relationship between diagnostic measurements obtained with the diagnostic medical device and use of the consumable;
obtaining, from the diagnostic medical device, diagnostic data pertaining to one or more diagnostic measurements;
employing the pre-determined correlation to calculate, based on the diagnostic data, an estimated rate of use of the consumable.

32. The computer-implemented method according to claim 31 wherein the diagnostic data includes risk factor data associated with a risk of contracting the medical condition.
